(19) United States
(12) Patent Application Publication
(10) Pub. No.: US 2010/0036473 A1
(43) Pub. Date: Feb. 11, 2010

(54) HEATABLE DELIVERY DEVICE

(75) Inventor: Noah Roth, Atlanta, GA (US)

Correspondence Address: FAY SHARPE LLP, 1228 Euclid Avenue, 5th Floor, The Halle Building, Cleveland, OH, 44115, US

(73) Assignee: ICON MEDICAL CORP., Atanta, GA (US)

(21) Appl. No.: 12/524,089

(22) PCT Filed: Jun. 20, 2008
(86) PCT No.: PCT/US2008/067700

§ 371 Date: Oct. 26, 2009

Related U.S. Application Data
(60) Provisional application No. 60/936,913, filed on Jun. 22, 2007.

(51) Int. Cl. A61F 2/06; A61F 7/00

(52) U.S. Cl. 623/1.11

(57) ABSTRACT

A deployment device is provided for delivery and placement of a polymeric implant and/or stent with a polymeric coating in a body passageway. The deployment device can be used to provide temperature controlled inflation fluid and/or temperature adjusted inflation fluid to locally heat the polymeric implant and or stent with a polymeric coating to achieve a relatively softer polymeric phase, reducing the risk of fracture of the polymeric implant and or stent with a polymeric coating during expansion.

The present invention claims priority on U.S. Provisional Patent Application Ser. No. 60/936,913 filed Jun. 22, 2007 entitled “HEATABLE DELIVERY DEVICE,” which is incorporated by reference in its entirety.

The present invention relates generally to medical devices, and particularly to a delivery device for use within a body, and more particularly to balloon and/or expansion devices used where local temperature control is desirable.

### BACKGROUND OF THE INVENTION

Medical treatment of various illnesses or diseases commonly includes the use of one or more medical devices. One type of medical device that is commonly used to repair various types of body passageways is an expandable stent. One purpose of a stent is to open a blocked or a partially blocked body passageway. The procedure of opening a blocked or a partially blocked body passageway commonly includes the use of one or more stents in combination with other medical devices such as, but not limited to, an introducer sheath, a guiding catheter, a guide wire, an angioplasty balloon, etc.

Various physical attributes of a stent can contribute directly to the success rate of the device. These physical attributes include radiopacity, hoop strength, radial force, thickness, dimensions and the like. Cobalt-Chromium and Stainless Steel are commonly used to form stents. These materials are commonly used since such materials have a known history of safety, effectiveness, and biocompatibility. Despite an initially successful dilation of the stent, vessel elastic recoil, thrombus formation, and smooth muscle proliferation contribute to the partial or full reclosure of the vessel following standard balloon angioplasty alone, and stenting with bare metal stents.

The prevailing therapy for diseased vessels includes the use of drug coated stents where the drug is targeted at further reducing the rate of restenosis. Concerns have developed over the long term safety of drug coated metallic stents. It is believed that if stents could be removed or absorbed after the period of acute vessel recoil and the local delivery drug that is targeted at reducing the rate of restenosis, such a procedure would result in a safer long term outcome.

However, bioabsorbable stents have limited physical performance as compared to more traditional metallic stents. More specifically, bioabsorbable stents need to have sufficient rigidity to overcome short term vessel recoil, but be malleable enough to be expanded without fracture.

The present invention is based upon the use of a delivery system having the capacity to locally heat a polymeric stent and/or polymeric stent coating to maintain arterial patency. Intravascular stents have been used for the purpose of improving luminal diameter, preventing abrupt reclosure of the vessel and reducing the incidence of restenosis after angioplasty. Conventional metallic stents have been found to be thrombogenic and inciting intimal hyperplasia. The current therapeutic regime relies on the use of drug coated stents to reduce the incidence of restenosis. However, recent studies have suggested an increase in the thrombogenecity associated with certain drug coated stents (when the drug has eluted out) as compared to bare metal stents. Bioabsorbable polymeric stents have sought to provide the advantages of drug delivery without the associated concerns of long term safety associated with an increase in thrombogenecity. Alternatively, it is the maintenance of thrombogenecity or lack of a decrease in thrombogenecity associated with the integration of bare metal stents that are thought to have contributed to the long term safety issues associated with bare metal stents.

Notwithstanding the prior art, there remains a need for a bioabsorbable stent delivery catheter with an inflation device that is able to temporarily increase and/or decrease the temperature of the bioabsorbable stent to facilitate expansion while not compromising the long term structural integrity of the stent.

### SUMMARY OF THE INVENTION

The present invention is generally directed to a medical device designed to temporarily and/or locally change the temperature of heat on another medical device so as to facilitate in the change in shape of the other medical device. More particularly, the medical device in accordance with the present invention is a delivery device or part of a delivery device for an expandable medical device such as, but not limited to, a stent. Even more particularly, the medical device in accordance with the present invention is a balloon catheter such as, but not limited to, a stent delivery catheter that is able to temporarily and/or locally heating a polymeric stent or a polymeric coating on the stent so as to make the stent softer during expansion. As can be appreciated, the present invention can be used to cool the stent after being heated and/or to control the temperature of the stent for some period of time. Although the present invention will make particular reference to stent delivery catheters, and will also be described in reference to a stent delivery catheter for use with a stent, it will be appreciated that the present invention has much broader application and can be associated with any type of medical device that requires a change in temperature during a certain type of medical procedure. As can also be appreciated, the present invention with make particular reference to polymeric stents and/or stents that include a polymeric coating; however, it will be appreciated that the present invention can be used with non-polymeric stents (e.g., metal stents, etc.) and/or stents that do not include a polymeric coating.

In accordance with one non-limiting aspect of the present invention, the medical device of the present invention can be in the form of a delivery catheter. The delivery catheter can be designed to at least partially include 1) a novel balloon that includes a conduit which can circulate fluid at a particular temperature, 2) a novel balloon that includes a conduit which can deliver fluid at a particular temperature and then expel such fluid in a body passageway, 3) an expansion device constructed of one or more polymeric and/or metallic bands that can be at least partially heated through electrical resistance and/or be heated fluid, and/or 4) an expansion device in the form of a braided balloon constructed using conductive braids that can be at least partially heated through electrical resistance and/or be heated fluid. As can be appreciated, the balloon can include other devices (e.g., fiberoptic wires, etc.) that can be used to cure and/or alter the chemical and/or physical properties of another medical device (e.g., cure poly on a stent, soften polymer on a stent, etc.). The delivery catheter can be used to provide a local change in temperature so as to heat a polymeric stent and/or stent coating above its glass transition temperature to facilitate in the expansion of the stent. The delivery catheter can also or alternatively be used to provide a local change in temperature so as to cool a polymeric stent and/or stent coating during and/or after the expansion of the stent. The modality of expansion associated with balloon expandable metallic stents include crimping the stent on a balloon delivery catheter, inserting the stent into the vasculature, maneuvering of the stent to the vessel being treated, expanding the balloon whereby the stent undergoes plastic deformation, making contact with, and maintaining the inner diameter of the expanded vessel segment, deflating the balloon catheter, and withdrawing the balloon delivery catheter from the human body. Conversely, polymeric stents and/or polymeric coated stents are more prone to fracture upon expansion and generally do not exhibit the expansion modality exhibited by metal stents. Namely, plastic deformation is generally accompanied by a predetermined amount of recoil. At temperatures below the glass transition temperature of the polymeric material, the polymeric material is less able to undergo deformation while maintaining structural form and integrity. The present invention provides a mechanism and/or process to locally heat a polymeric stent and/or polymeric stent coating to a temperature that is closer to, at, or greater than the glass transition temperature of the polymer so as to facilitate in the expansion of polymeric stents and/or polymeric coated stents. Once the polymeric stents and/or polymeric coated stents are expanded, the polymeric stents and/or polymeric coated stents are then allowed to cool by removing heat from the stent (e.g, applying a cooling fluid to the stent, etc.) and/or by natural locally cooling of the stent below its glass transition temperature while positioned in the body passageway. The polymeric stents and/or stent coatings can be viewed as hardened material requiring a secondary process to assist in the deformation process. Thermoplastic materials which are initially solid cab become softened and moldable when heated to their glass transition temperature. Still another advantage of locally heating a polymeric stent and/or polymeric coated stent above its glass transition temperature is the ability to better match the contour and diameter of the body passageway when the stent is expanded in the body passageway.

In accordance with another and/or additional non-limiting aspect of the present invention, the present invention relates generally to devices and methods for medical treatment, and more particularly to angioplasty and improvements in a method and apparatus for preventing restenosis after treatment. More particularly the present invention relates to stent delivery catheters inserted into the body and expansion of polymeric stents and/or metallic stents with polymeric coatings. Alternatively and/or additionally, the invention relates to devices and methods associated with local oblation therapy within the vasculature.

In accordance with still another and/or additional non-limiting aspect of the present invention, the present invention is generally directed to a delivery and/or an expansion device that is able to a) at least partially locally heat a polymeric bioabsorbable and/or polymeric biodegradable device above a glass transition temperature of the polymeric bioabsorbable and/or polymeric biodegradable device, and/or b) at least partially heat a polymeric coating above a glass transition temperature of the polymeric coating, which polymeric coating is positioned at least partially on a bioabsorbable device and/or biodegradable device or on a non-bioabsorbable and/or a non-biodegradable device. As can be appreciated, the delivery and/or an expansion device can, but is not required, to be able to a) at least partially locally cool a polymeric bioabsorbable and/or polymeric biodegradable device below a glass transition temperature of the polymeric bioabsorbable and/or polymeric biodegradable device, and/or b) at least partially cool a polymeric coating below a glass transition temperature of the polymeric coating, which polymeric coating is positioned at least partially on a bioabsorbable device and/or biodegradable device or on a non-bioabsorbable and/or a non-biodegradable device. The heating of the polymeric coating, the polymeric bioabsorbable and/or the polymeric biodegradable device can 1) facilitate in the delivery and/or expansion of the bioabsorbable device and/or biodegradable device or non-bioabsorbable and/or non-biodegradable device in a body passageway, and/or 2) minimize or prevent fracture of the polymeric coating, the polymeric bioabsorbable and/or the polymeric biodegradable device during expansion of the polymeric bioabsorbable device, the polymeric biodegradable device, and/or the device that at least partially includes the polymeric coating. In one non-limiting embodiment of the invention, the delivery and/or expansion device of the present invention can be in the form of a delivery balloon with a re-circulating fluid path and/or a one way fluid path whereby fluid at a particular temperature can be directed into the delivery and/or expansion device to locally heat/cool the polymeric bioabsorbable device and/or polymeric biodegradable device, and/or polymeric coating on the bioabsorbable device, the biodegradable device, the non-bioabsorbable device, and/or the non-biodegradable device above/below the polymeric material glass transition temperature. The heating and/or cooling of the bioabsorbable device and/or polymeric biodegradable device, and/or polymeric coating on the bioabsorbable device, the biodegradable device, the non-bioabsorbable device, and/or non-biodegradable device can be in a continuous or noncontinuous manner. In another and/or additional non-limiting embodiment of the invention, the delivery and/or expansion device of the present invention can include an expansion component (e.g., balloon, etc.) wherein the heated/cooled fluid is used to at least partially expand and/or at least partially heat/cool the expansion component to a particular temperature. The heating/cooling of the expansion component can be used to heat/cool the polymeric bioabsorbable device and/or polymeric biodegradable device, and/or polymeric coating on the bioabsorbable device, the biodegradable device, the non-bioabsorbable device, and/or non-biodegradable device above/below the polymeric material glass transition temperature. The heating/cooling of the polymeric material can occur prior to, during, and/or after the partial or full expansion of the polymeric bioabsorbable device and/or polymeric biodegradable device, and/or polymeric coating on the bioabsorbable device, the biodegradable device, the non-bioabsorbable device, and/or the non-biodegradable device. In still another and/or additional non-limiting embodiment of the invention, the delivery and/or expansion device of the present invention can include one or more heat conducting regions or bands that can be used to at least partially heat the polymeric bioabsorbable device and/or polymeric biodegradable device, and/or polymeric coating on the bioabsorbable device, the biodegradable device, the non-bioabsorbable device, and/or the non-biodegradable device above the polymeric material glass transition temperature. The one or more heat conducting regions or bands can be designed to be at least partially heated by electric resistance heating; however, this is not required. The one or more heat conducting regions or bands can be designed of a polymeric material with a metallic center and/or metallic material able to be heated; however, other or additional configurations can be used. The one or more heat conducting regions or bands can be used in conjunction with, or independent from an expansion device (e.g., balloon, etc.). In yet another and/or additional non-limiting embodiment of the invention, the delivery and/or expansion device of the present invention can include a heat conductive braided and/or meshed structure. This heat conductive braided and/or meshed structure can be positioned inside and/or about an expansion device (e.g., balloon, etc.). The heat conductive braided and/or meshed structure can be used to at least partially heat the polymeric bioabsorbable device and/or polymeric biodegradable device, and/or polymeric coating on the bioabsorbable device, the biodegradable device, the non-bioabsorbable device, and/or the non-biodegradable device above the polymeric material glass transition temperature. As can be appreciated, the heat conductive material can be used independently or in conjunction with a heat fluid and/or cooling fluid.

In accordance with yet another and/or additional non-limiting aspect of the present invention, the present invention can be used in conjunction with many different devices such as, but not limited to, a stent, an endovascular graft, a surgical graft (e.g., vascular graft, etc.), polymeric scaffolds for tissue regeneration, etc. These non-limiting devices can be partially or fully formed and/or coated with one or more types of polymeric materials that are required to undergo expansion and/or deflection while in use.

In accordance with still yet another and/or additional non-limiting aspect of the present invention, the medical device of the present invention is directed for use in a body passageway. As used herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, subarachnoid space, and central and peripheral nerve conduits, etc.). The techniques employed to deliver the device to a treatment area include, but are not limited to, angioplasty, vascular anastomoses, transplantation, implantation, surgical implantation, subcutaneous introduction, minimally invasive surgical procedures, interventional procedures, and any combinations thereof. For vascular applications, the term “body passageway” primarily refers to blood vessels and chambers in the heart.

In accordance with another and/or additional non-limiting aspect of the present invention, the polymeric device and/or polymeric coating can include, contain and/or be coated with one or more chemical agents that are used to facilitate in the success of the device and/or treatment area. The term “chemical agent” includes, but is not limited to, a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by the one or more chemical agents include, but are not limited to viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. Non-limiting examples of chemical agents that can be used include, but are not limited to, an anti-platelet compound and/or anticoagulant compound such as, but not limited to, warfarin (Coumadin), warfarin derivatives, aspirin, aspirin derivatives, clopidogrel, clopidogrel derivatives, ticlopadine, ticlopadine derivatives, hirdun, hirdun derivatives, dipyridamole, dipylidamole derivatives, trapidil, trapidil derivatives, taxol, taxol derivatives, cytochalasin, cytochalasin derivatives, paclitaxel, paclitaxel derivatives, rapamycin, rapamycin derivatives, GM-CSF, GM-CSF derivatives, heparin, heparin derivatives, low molecular weight heparin, low molecular weight heparin derivatives, and combinations thereof. One specific non-limiting example of an anti-thrombotic inhibitor that can be included with, contained in and/or be coated on the polymeric bioabsorbable device and/or polymeric biodegradable device and/or polymeric coating on the bioabsorbable device, biodegradable device, non-bioabsorbable device, non-biodegradable device includes 1) huridin and/or derivatives, and/or 2) alagors (e.g., bivalirudin, etc.) and/or derivatives. As can be appreciated, one or more other anti-thrombotic chemical agents can be used with the polymeric bioabsorbable device and/or polymeric biodegradable device, and/or polymeric coating on the bioabsorbable device, the biodegradable device, the non-bioabsorbable device, and/or the non-biodegradable device. Non-limiting examples of chemical agents that can be used include, but are not limited to, 5-Fluorouracil and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; chemical agents that modulate intracellular Ca2+ transport such as L-type (e.g., diltiazem, nifedipine, verapamil, etc.) or T-type Ca2+ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; antibiotics; anti-coagulant compounds and/or derivatives thereof; anti-fibrosis compounds and/or derivatives thereof; antifungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., fortyrosine kinase, protein kinase C, myosin light chain kinase, Ca2+/calmodulin kinase II, casein kinase II, etc.); aspirin and/or derivatives thereof; azathioprine and/or derivatives thereof; β-Estradiol and/or derivatives thereof; β-1-anticollagenase and/or derivatives thereof; calcium channel blockers and/or derivatives thereof; calmodulin antagonists and/or derivatives thereof (e.g., H7, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP3 receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-1 and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H3P32O4, etc.); rapamvcin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; TH1 and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the chemical agent can include one or more derivatives of the above listed compounds and/or other compounds. In one non-limiting embodiment, the chemical agent includes, but is not limited to, trapidil, Trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, GM-CSF (granulo-cytemacrophage colony-stimulating-factor), GM-CSF derivatives, statins or HMG-CoA reductase inhibitors forming a class of hypolipidemic agents, combinations, or analogs thereof, and combinations thereof. The type and/or amount of chemical agent included in the polymeric bioabsorbable device and/or polymeric biodegradable device, and/or polymeric coating on the bioabsorbable device, the biodegradable device, the non-bioabsorbable device, and/or the non-biodegradable device; and/or coated on the polymeric bioabsorbable device and/or polymeric biodegradable device, and/or polymeric coating on the bioabsorbable device, the biodegradable device, the non-bioabsorbable device, and/or the non-biodegradable device can vary. When two or more chemical agents are included in and/or coated on the polymeric bioabsorbable device and/or polymeric biodegradable device, and/or polymeric coating on the bioabsorbable device, the biodegradable device, the non-bioabsorbable device, and/or the non-biodegradable device, the amount of two or more chemical agents can be the same or different. The type and/or amount of chemical agent included on, in and/or in conjunction with the polymeric bioabsorbable device and/or polymeric biodegradable device, and/or polymeric coating on the bioabsorbable device, the biodegradable device, the non-bioabsorbable device, and/or the non-biodegradable device are generally selected to address one or more clinical events. Typically, the amount of chemical agent included on, in and/or used in conjunction with the polymeric bioabsorbable device and/or polymeric biodegradable device, and/or polymeric coating on the bioabsorbable device, the biodegradable device, the non-bioabsorbable device, and/or the non-biodegradable device is about 0.01-100 ug per mm² and/or at least about 0.01 weight percent of polymeric bioabsorbable device and/or polymeric biodegradable device, and/or polymeric coating on the bioabsorbable device, the biodegradable device, the non-bioabsorbable device, and/or non-biodegradable device; however, other amounts can be used. In one non-limiting embodiment of the invention, the polymeric bioabsorbable device and/or polymeric biodegradable device, and/or polymeric coating on the bioabsorbable device, the biodegradable device, the non-bioabsorbable device, and/or the non-biodegradable device can be partially or fully coated and/or impregnated with one or more chemical agents to facilitate in the success of a particular medical procedure. The amount of two or more chemical agents on, in and/or used in conjunction with the polymeric bioabsorbable device and/or polymeric biodegradable device, and/or polymeric coating on the bioabsorbable device, the biodegradable device, the non-bioabsorbable device, and/or the non-biodegradable device can be the same or different. The one or more chemical agents can be coated on and/or impregnated in the polymeric bioabsorbable device and/or polymeric biodegradable device, and/or polymeric coating on the bioabsorbable device, the biodegradable device, the non-bioabsorbable device, and/or the non-biodegradable device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the invention, the type and/or amount of chemical agent included on, in and or in conjunction with the polymeric bioabsorbable device and/or polymeric biodegradable device, and/or polymeric coating on the bioabsorbable device, the biodegradable device, the non-bioabsorbable device, and/or the non-biodegradable device is generally selected for the treatment of one or more clinical events. Typically, the amount of chemical agent included on, in and/or used in conjunction with the polymeric bioabsorbable device and/or polymeric biodegradable device, and/or polymeric coating on the bioabsorbable device, the biodegradable device, the non-bioabsorbable device, and/or the non-biodegradable device are about 0.01-100 ug per mm² and/or at least about 0.01-100 weight percent of the polymeric bioabsorbable device and/or polymeric biodegradable device, and/or polymeric coating on the bioabsorbable device, the biodegradable device, the non-bioabsorbable device, and/or the non-biodegradable device; however, other amounts can be used. The amount of two or more chemical agents on, in and/or used in conjunction with the polymeric bioabsorbable device and/or polymeric biodegradable device, and/or polymeric coating on the bioabsorbable device, the biodegradable device, the non-bioabsorbable device, the non-biodegradable device can be the same or different. For instance, portions of the polymeric bioabsorbable device and/or polymeric biodegradable device, and/or polymeric coating on the bioabsorbable device, the biodegradable device, the non-bioabsorbable device, and/or the non-biodegradable device to provide local and/or systemic delivery of one or more chemical agents in and/or to a body passageway to a) inhibit or prevent thrombosis, in-stent restenosis, vascular narrowing and/or restenosis after the polymeric bioabsorbable device and/or polymeric biodegradable device, and/or polymeric coating on the bioabsorbable device, the biodegradable device, the non-bioabsorbable device, and/or the non-biodegradable device has been inserted in and/or connected to a body passageway, b) at least partially passivate, remove, encapsulate, and/or dissolve lipids, fibroblast, fibrin, etc. in a body passageway so as to at least partially remove such materials and/or to passivate such vulnerable materials (e.g., vulnerable plaque, etc.) in the body passageway in the region of the polymeric bioabsorbable device and/or polymeric biodegradable device and/or, polymeric coating on the bioabsorbable device, the biodegradable device, the non-bioabsorbable device, and/or the non-biodegradable device, and/or downstream of the polymeric bioabsorbable device and/or polymeric biodegradable device, and/or polymeric coating on the bioabsorbable device, the biodegradable device, the non-bioabsorbable device, and/or the non-biodegradable device. As can be appreciated, the one or more chemical agents can have many other or additional uses. In still another and/or alternative non-limiting example, the polymeric bioabsorbable device and/or polymeric biodegradable device, and/or polymeric coating on the bioabsorbable device, the biodegradable device, the non-bioabsorbable device, and/or the non-biodegradable device is coated with, and/or includes one or more chemical agents such as, but not limited to chemical agents associated with thrombolytics, vasodilators, anti-hypertensive agents, antimicrobial or anti-biotic, anti-mitotic, anti-proliferative, anti-secretory agents, non-steroidal anti-inflammatory drugs, immunosuppressive agents, growth factors and growth factor antagonists, endothelial growth factors and growth factor antagonists, antitumor and/or chemotherapeutic agents, anti-polymerases, anti-viral agents, anti-body targeted therapy agents, hormones, anti-oxidants, biologic components, radio-therapeutic agents, radiopaque agents and/or radio-labeled agents. In addition to these chemical agents, the polymeric bioabsorbable device and/or polymeric biodegradable device, and/or polymeric coating on the bioabsorbable device, the biodegradable device, the non-bioabsorbable device, and/or the non-biodegradable device can be coated with and/or include one or more chemical agents that are capable of inhibiting or preventing any adverse biological response by and/or to the device that could possibly lead to device failure and/or an adverse reaction by human or animal tissue. A wide range of chemical agents thus can be used. The one or more chemical agents can be coated on and/or impregnated in the polymeric bioabsorbable device and/or polymeric biodegradable device and/or polymeric coating on the bioabsorbable device, the biodegradable device, the non-bioabsorbable device, and/or the non-biodegradable device by a variety of mechanisms such as, but not limited to, spraying (e.g., atomizing spray techniques, etc.), dip coating, roll coating, sonication, brushing, plasma deposition, depositing by vapor deposition.

In accordance with still another and/or additional non-limiting aspect of the present invention, the polymeric device and/or polymeric coatings can be formed of a biodegradable polymer that includes, but is not limited to, parylene, PLGA, POE, PGA, PLLA, PAA, PEG, chitosan and/or copolymers, blends, and/or composites of above and/or derivatives of one or more of these polymers. In one non-limiting embodiment of the invention, the polymeric device includes a body of which a majority is formed of a biodegradable polymer system and that at least a portion of the body includes and/or is coated with a nonporous polymer that includes, but is not limited to, polyamide, parylene c, parylene n and/or a parylene derivative. In another and/or alternative non-limiting embodiment of the invention, the polymeric device includes a body of which a majority is formed of a biodegradable polymer system and that at least a portion of the body includes and/or is coated with poly(ethylene oxide), poly(ethylene glycol), and poly(propylene oxide), polymers of silicone, methane, tetrafluoroethylene (including TEFLON brand polymers), tetramethyldisiloxane, and the like.

In accordance with yet another and/or additional non-limiting aspect of the present invention, the expansion device is designed such that heated fluid can be used to at least partially heat and expand the expansion device (e.g., balloon, etc.). For example, when the expansion device includes and/or is in the form of a balloon, the heated fluid used to expand the balloon also heats the surface of the balloon. During expansion of the balloon, the heated balloon surface contacts the polymeric stent and/or polymeric stent coating and heats the polymeric stent and/or polymeric stent coating via conduction. The temperature of the fluid (e.g., heated water, heated saline solution, etc.) can be used to control the temperature of the polymeric stent and/or polymeric stent coating prior to, during and/or after the polymeric stent and/or polymeric stent coating is expanded in a body passageway. As such, the polymeric stent and/or polymeric stent coating can be heated, cooled, reheated, recooled, etc. by the temperature controlled fluid that flows into the balloon. In one non-limiting methodology, the surface of the balloon and subsequently, the polymeric stent and/or polymeric stent coating are cooled (slowly or rapidly) by replacing the heated fluid with a fluid either at room temperature or some temperature below room temperature. As can be appreciated, the temperature of the inflation balloon, polymeric stent, and/or polymeric stent coating can be reduced without the use of a cold inflation fluid. For instance, the heat from the inflation balloon, polymeric stent, and/or polymeric stent coating can be transferred to the surrounding tissue and flowing blood thereby bringing the polymeric stent, and/or polymeric stent coating into equilibrium with its surroundings.

In accordance with still yet another and/or additional non-limiting aspect of the present invention, heated and/or cooled fluid can be continually injected through an inflation port of the expansion device (e.g., balloon, etc.) and be subsequently discharged from the expansion device into a body passageway at a rate that is required to obtain the desired temperature of the polymeric stent and/or polymeric stent coating. Continuous fluid injection into the expansion device can be accomplished by the use of a number of devices such as, but not limited to, a syringe, an automatic syringe injector, an endoflator, a power injector, or by some alternate means.

In accordance with another and/or additional non-limiting aspect of the present invention, the heated and/or cooled fluid can be continually injected through an inflation port of the expansion device (e.g., balloon, etc.) and be subsequently returned so as to create a continuous flow loop for the fluid. The fluid can be thus continually recirculated through the expansion device to obtain the desired temperature of the polymeric stent and/or polymeric stent coating. The flow rate of the fluid into and/or out of the expansion device can be held constant or varied. The fluid injection into and/or out of the expansion device can be accomplished by the use of a number of devices such as, but not limited to, a syringe, an automatic syringe injector, an endoflator, a power injector, or by some alternate means. In a closed loop system, the same fluid is continually used throughout the process. The fluid can be heated and/or cooled multiple times. The heating and/or cooling of the fluid can be accomplished external to and/or internally in the body passageway. A sufficient pressure differential between the supply and return port in the expansion device is generally necessary to maintain and effectuate the desired balloon inflation and/or deflation. A fluid reservoir that is located externally to the body passageway can be used; however, this is not required. One or more valves, flow controllers, orifices, etc. can be used to at least partially control the flow rate of the fluid to and/or from the expansion device; however, this is not required.

In accordance with still another and/or additional non-limiting aspect of the present invention, the heated/cooled fluid can be radioactive. The radioactive fluid can be used to at least partially cause cross linking of the polymer structure of the polymeric stent and/or polymeric coating so as to cause the stent and/or coating on the stent to become more rigid in its form. The polymeric stent or polymeric coated stent can still be expanded as described previously with the use of heat; however, upon expansion, the radioactive fluid can be introduced to cross-link the polymer matrix helping it to resist acute vessel recoil. In addition, another and/or alternative non-limiting aspect of the use of radioactive fluids includes the initiation of the absorption of a bioabsorbable stent.

In accordance with still another and/or additional non-limiting aspect of the present invention, the heated/cooled fluid can include one or more chemical agents. When the heated/cooled fluid is at least partially released into the body passageway, the chemical agents in the fluid are also released into the body passageway. As such, controlled amounts of one or more chemical agent can be released into the body passageway prior to, during and/or after the insertion of the polymeric stent and/or polymeric coated stent.

In accordance with yet another and/or additional non-limiting aspect of the present invention, the expansion device can include a heat conductive material such as, but not limited to, a braid and/or mesh material. In one non-limiting embodiment of the invention, the expansion device includes a balloon and a metal wire braid along the radius of the balloon. The braided metal wire is generally positioned inside the balloon and in the form of a tube; however, this is not required. The braided metal wire can be insulated to prevent thermal damage to the catheter body and/or to the balloon; however, this is not required. The braided wire can be heated by electrical and/or RF energy that is supplied through one or more thin wires which extend along the length of the catheter to the balloon. As can be appreciated, the braided wire can be also or alternatively heated by a heated fluid and/or by some external means (e.g., external electromagnetic waves, etc.). This arrangement can be used in conjunction with heated fluid to facilitate in maintaining the fluid temperature within the balloon. Alternatively, this arrangement can be used to fully heat the fluid in the balloon and/or polymeric stent and/or polymeric coated stent. In another and/or additional non-limiting embodiment of the invention, the braided or mesh wire arrangement includes proximal ends of the wires that can be used to connect to an energy source producing a current source, which current source causes the braided or mesh wire to heat which in turn causes the balloon surface to be heated. As can be appreciated, the heat conductive material can also be designed to convey electromagnetic waves (e.g., IR light, UV light, etc.); however, this is not required. The electromagnetic waves, when conveyed to the balloon, can be used to 1) cure one or more polymers on the stent, 2) begin the degradation process on the stent, 3) soften the polymer on the stent, 4) harden the polymer on the stent, etc.

The invention may best be understood with reference to the accompanying drawings wherein an illustrative embodiment is shown. Further aspects and advantages of the present invention will be recognized and understood by those of skill in the art upon reading of the detailed description and examples of the invention set forth here and in the accompanying drawings. The invention is applicable to both monorail and rapid exchange balloon catheter systems.

### BRIEF DESCRIPTION OF THE DRAWINGS

Reference may now be made to the drawings, which illustrate various non-limiting embodiments that the invention may take in physical form and in certain parts and arrangements of parts wherein:

FIG. 1 is a side cross-sectional view of a recirculating inflation balloon showing an inflation balloon in the expanded state and supply and return ports;

FIG. 2 is a side cross-sectional view of a coaxal inflation balloon where the heated and/or cooled fluid is injected and aspirated through the same inflation zone;

FIG. 3 is a side cross-sectional view of a coaxal inflation balloon where the heated and/or cooled fluid is injected through one inflation zone and discharge into the vessel through distally located discharge ports in the balloon inner assembly;

FIG. 4 is a side cross-sectional view of a coaxal inflation balloon system containing a braiding in the balloon and two wires extending proximally connected to a current and/or RF generator;

FIG. 5 is a view of the braining in the braided balloon structure showing the braided wires extending around the balloon structure in a helical pattern;

FIG. 6 is a view of a braided balloon structure showing the braided wires extending around the balloon structure in a helical pattern and containing undulations to increase the resistance and thereby heat generation as current and/or RF energy passes through the braided wire;

FIG. 7 is a view of a delivery catheter which balloon comprises a helical band with an inflation and deflation port;

FIG. 8 is a view of a delivery catheter which balloon comprises a helical band with an inflation and deflation port in an expanded state;

FIG. 9 is a perspective view of a balloon catheter that includes several different ports that can be used to control the delivery and expansion of a stent in a body passageway;

FIG. 10 is a perspective view of a balloon catheter that includes several different ports that can be used to control the delivery and expansion of a stent in a body passageway and the balloon includes a wire braid inside the balloon;

FIG. 11 is an end view of the balloon catheter of FIG. 10 that illustrates the various connection ports and openings for the balloon catheter;

FIG. 12 is a cross-section view of a balloon that includes a metal braid; and,

FIG. 13 is a cross-section view of a balloon disclosing inlet and outlet fluid openings in the balloon.

### DETAILED DESCRIPTION OF THE INVENTION

Referring now to the drawings wherein the showings are for the purpose of illustrating non-limiting embodiments of the invention only and not for the purpose of limiting the same, FIGS. 1-13 disclose several non-limiting deployment devices in accordance with the present invention. The deployment devices illustrated in FIGS. 1-13 are used to deliver an expandable device such as, but not limited to, a polymeric medical device and/or a medical device having a polymeric coating to a region in a body passageway.

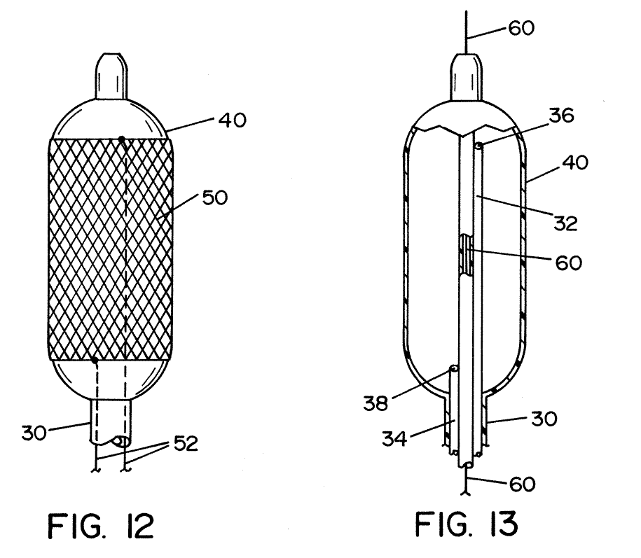

Referring now to FIG. 1, there is illustrated a side cross-sectional view of an end portion of a balloon catheter 20. An inflatable balloon 40 is secured to the end region of the catheter 30. The design and use of catheters for delivery of medical devices such as stents in a body passageway are well known in the art, thus will not be described in detail herein. The catheter 30 includes two channels 32, 34 that enable fluid to flow therethrough; however, it will be appreciated that more than two channels can be used or only one channel can be used. Each channel includes an opening 36, 38, which openings are positioned in the interior of balloon 40. As illustrated by the arrows, channel 34 is designed to convey fluid to the balloon and to at least partially fill the balloon with fluid via opening 38. Channel 32 is designed to convey fluid from the balloon via opening 36. By controlling the rate of fluid flow into and out of the balloon, the degree of inflation of the balloon can be controlled. The flow of fluid to the balloon can be achieved in a variety of ways such as, but not limited to, the use of a syringe, an automatic syringe injector, an endoflator, a power injector, or by some alternate means. The surface temperature of the balloon can be at least partially controlled by the temperature of the fluid being conveyed into the balloon. The surface temperature of the balloon can then be used to heat/cool one or more polymers that at least partially form a medical device and/or that are coated on a medical device that is at least partially mounted on the balloon. The arrangement illustrated in FIG. 1 enables fluid to be recirculated through the balloon. Referring now to FIG. 13, a cross-section view of balloon 40 is illustrated. This cross-section illustrates in more detail one non-limiting arrangement for the channels 32, 34 and opening 36, 38 in the interior of the balloon. Opening 36 represents an inlet lumen opening inside the balloon and opening 38 represents an outlet lumen opening inside the balloon. The cross-section also illustrates a guide wire 60 which is well known in the art, thus will not be further described.

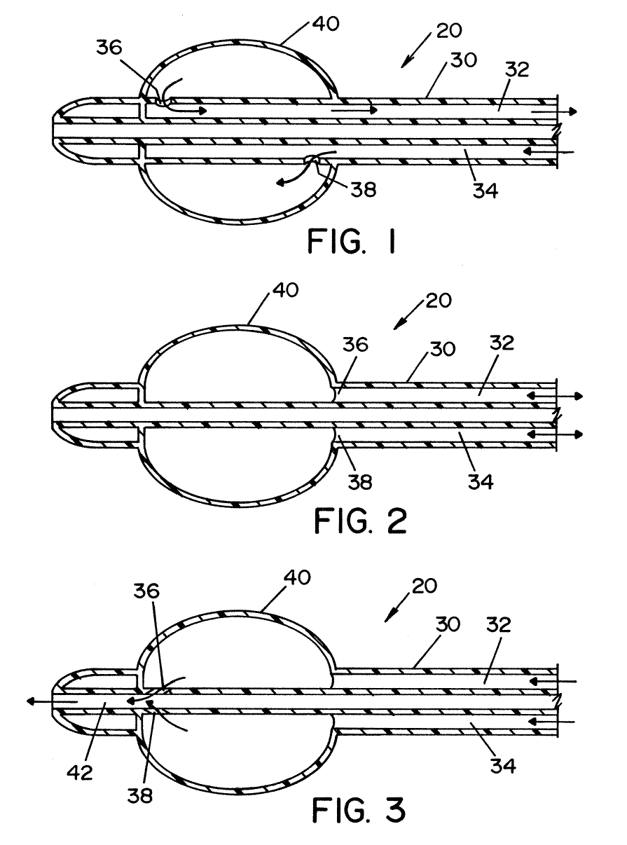

Referring now to FIG. 2, there is illustrated a side cross-sectional view of an end portion of a balloon catheter 20. An inflatable balloon 40 is secured to the end region of the catheter 30. The catheter 30 includes two channels 32, 34 that enable fluid to flow therethrough; however, it can be appreciated that only one channel can be used or more than two channels can be used. Each channel includes an opening 36, 38 which openings are positioned in the interior of the balloon 40. As illustrated by the arrows, channels 34, 36 are designed to either convey fluid to the balloon and at least partially fill the balloon with fluid via openings 36, 38 or convey fluid from the balloon via openings 36, 38. By controlling the amount of fluid into and/or out of the balloon, the degree of inflation of the balloon can be controlled. The flow of fluid to the balloon can be achieved in a variety of ways such as, but not limited to, the use of a syringe, an automatic syringe injector, an endoflator, a power injector, or by some alternate means. The surface temperature of the balloon can be at least partially controlled by the temperature of the fluid being conveyed into the balloon. The surface temperature of the balloon can then be used to heat/cool one or more polymers that at least partially form a medical device and/or that are coated on a medical device that is at least partially mounted on the balloon.

Referring now to FIG. 3, there is illustrated a side cross-sectional view of an end portion of a balloon catheter 20. An inflatable balloon 40 is secured to the end region of the catheter 30. The catheter 30 includes two channels 32, 34 that enable fluid to flow therethrough; however, it can be appreciated that only one channel can be used or more than two channels can be used. Each channel includes an opening 36, 38 which openings are positioned in the interior of the balloon 40. As illustrated by the arrows, channels 34, 36 are designed to convey fluid to the balloon and at least partially fill the balloon with fluid via openings 36, 38. The fluid in the balloon can exit the balloon via opening 42 and into the body passageway. By controlling the amount of fluid into and/or out of the balloon, the degree of inflation of the balloon can be controlled. The flow of fluid to the balloon can be achieved in a variety of ways such as, but not limited to, the use of a syringe, an automatic syringe injector, an endoflator, a power injector, or by some alternate means. The surface temperature of the balloon can be at least partially controlled by the temperature of the fluid being conveyed into the balloon. The surface temperature of the balloon can then be used to heat/cool one or more polymers that at least partially form a medical device and/or that are coated on a medical device that is at least partially mounted on the balloon.

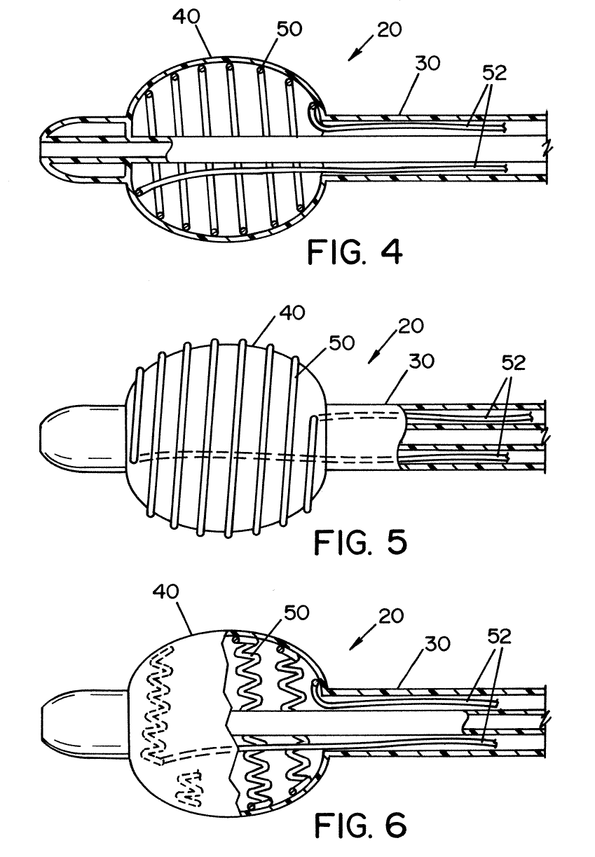

Referring now to FIG. 4, there is illustrated a side cross-sectional view of an end portion of a balloon catheter 20. An inflatable balloon 40 is secured to the end region of the catheter 30. The catheter 30 includes one or more channels to enable a fluid to inflate and/or deflate the balloon. The channels can be the same or similar to the channels discussed above with regard to FIGS. 1-3; however, this is not required. The one or more channels can direct a temperature controlled fluid to the balloon as discussed above; however, this is not required. By controlling the amount of fluid into and/or out of the balloon, the degree of inflation of the balloon can be controlled. The flow of fluid to the balloon can be achieved in a variety of ways such as, but not limited to, the use of a syringe, an automatic syringe injector, an endoflator, a power injector, or by some alternate means. The fluid can be a liquid and/or a gas. The surface temperature of the balloon can be at least partially controlled by the one or more wires 50 positioned inside the balloon. Through resistive and/or conductive heating via the one or more wires 50, the outer surface of the balloon can be controllably heated. Heated/cooled fluid as described above can also be directed to the interior of the balloon to controllably heat/cool the outer surface of the balloon; however, this is not required. One or more of the wires can be insulated to protect the balloon from damage; however, this is not required. Ends 52 of one or more of the wires can be connected to a heat and/or power source (e.g., AC source, DC source, RF generator, etc.), not shown, via one of more leads. The one or more leads can be positioned in one or more channels in the catheter. The surface temperature of the balloon can then be used to heat/cool one or more polymers that at least partially form a medical device and/or that are coated on a medical device that is at least partially mounted on the balloon.

Referring now to FIGS. 5 and 6, there are illustrated side views of two different wire configurations that can be positioned in the balloon. As can be appreciated, many other wire configurations can be used. As shown in FIG. 5, the wires in the balloon are in the form of a wire braid. FIG. 6 illustrates the wires in the balloon as a wire mesh and/or wire rings. Referring now to FIG. 12, there is illustrated a cross-sectional view of a balloon 40 that includes braided wires 50 in the interior of the balloon. Two lead wires 52 extend out one end of the balloon and are connected to a heat and/or power source, not shown.

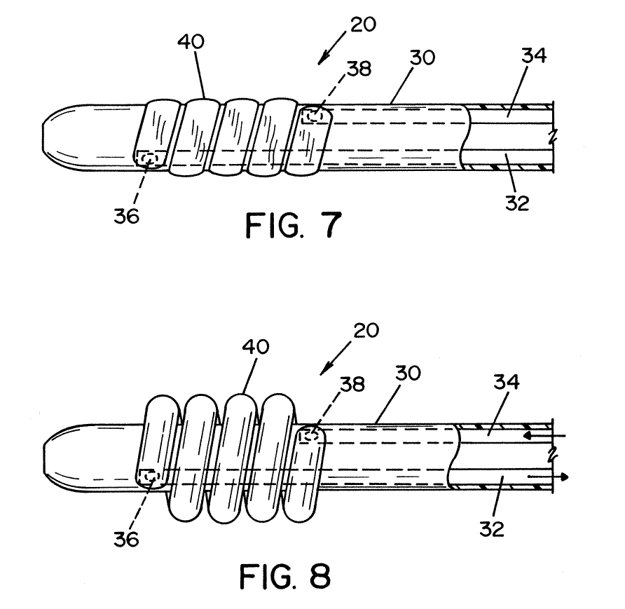

Referring now to FIGS. 7-8, there are illustrated side views of an end portion of a balloon catheter 20. An inflatable balloon 40 having a spiral or helical configuration is secured to the end region of the catheter 30. The catheter 30 includes two channels 32, 34 that enable fluid to flow therethrough; however, it will be appreciated that more than two channels can be used or only one channel can be used. Each channel includes an opening, not shown, which openings are positioned in the interior of the balloon 40. As illustrated by the arrows, channel 34 is designed to convey fluid to the balloon and at least partially fill the balloon with fluid via opening 38. Channel 32 is designed to convey fluid from the balloon via opening 36. By controlling the rate of fluid flow into and out of the balloon, the degree of inflation of the balloon can be controlled. The flow of fluid to the balloon can be achieved in a variety of ways such as, but not limited to, the use of a syringe, an automatic syringe injector, an endoflator, a power injector, or by some alternate means. The surface temperature of the balloon can be at least partially controlled by the temperature of the fluid being conveyed into the balloon. The surface temperature of the balloon can then be used to heat/cool one or more polymers that at least partially form a medical device and/or that are coated on a medical device that is at least partially mounted on the balloon. The arrangement illustrated in FIG. 7 enables fluid to be recirculated through the balloon. FIG. 7 illustrates the balloon in an uninflated condition and FIG. 8 illustrates the balloon in a partial or full inflated condition. The balloon can include one or more wires as described above with regard to FIGS. 4-6; however, this is not required. As can also be appreciated, balloon as described in FIGS. 1-3 can also include one or more wires as described above with regard to FIGS. 4-6; however, this is not required.

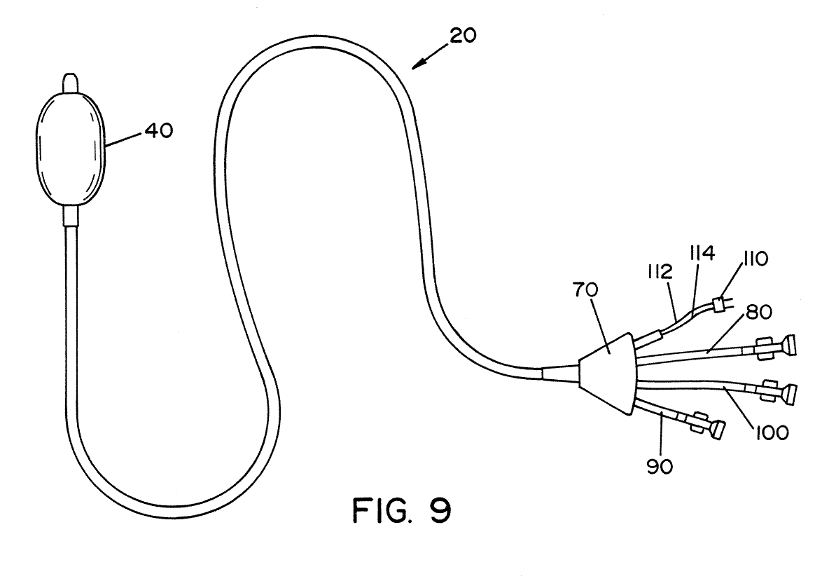

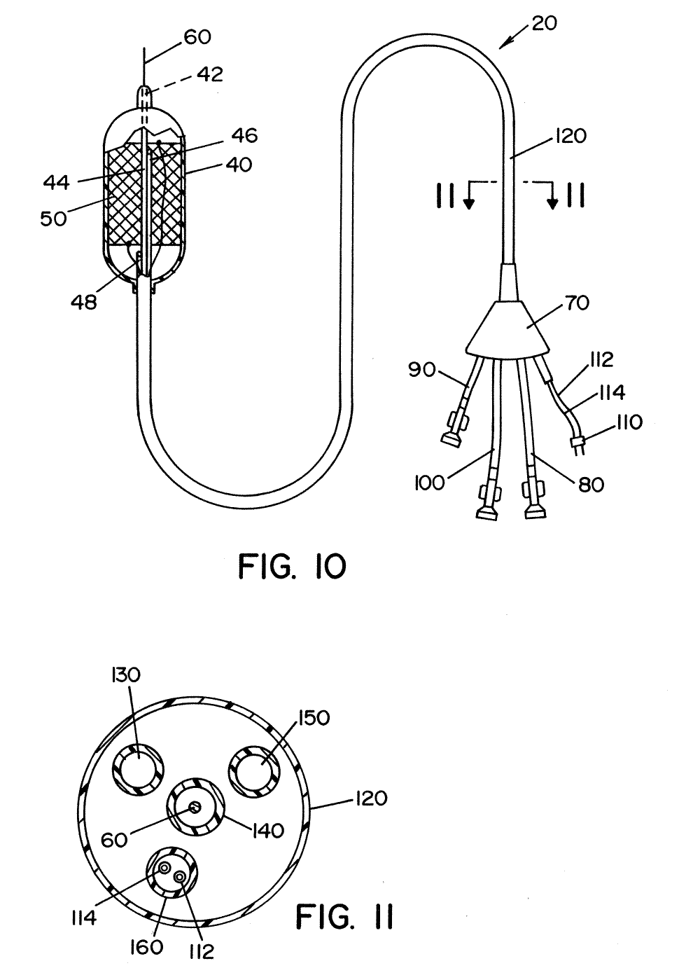

Referring now to FIGS. 9-11, there is illustrated a balloon catheter 20 in accordance with the present invention. FIG. 9 illustrated a balloon catheter that includes a balloon 40 positioned at one end and a system of connectors and ports at the other end. FIG. 10 illustrates a partial sectional view of the balloon 40 that has a braided wire 50 in the interior of the balloon. The end portion of the balloon can have a guide wire port 42; however, this is not required. The use of guide wire ports in balloons is well known in the art, thus will not be described herein. The balloon can include a guide wire tube 44 for a guide wire 60 that is positioned in the interior of the balloon. The balloon includes an inlet opening 46 to allow fluid to expand the balloon. The balloon can also include an outlet opening 48 to enable fluid to escape from the balloon; however, this is not required. The end portion 70 of the catheter is illustrated as including three ports, namely a guide wire port 80, a fluid inlet port 90, and a fluid outlet port 100. If fluid is to not be recirculated through the balloon, one of the fluid ports can be eliminated. Furthermore, the single fluid port can function as both the fluid inlet/outlet port. As can be appreciated, more than two fluid ports can be used. The guide wire port, which is well known in the art, is used to insert a guide wire 60 into the interior of the catheter so as to guide a medical device, not shown, that is positioned at least partially on the balloon to a particular region in a body passageway. The fluid inlet and outlet ports are used to control the flow of fluid to the balloon to inflate and/or deflated the balloon. Temperature controlled fluid can be flowed through the fluid inlet and/or outlet ports to control the temperature of the balloon as discussed above. The end portion of the balloon catheter also includes an electrical connector 110. When the balloon does not include one or more wires 50, the electrical connector can be eliminated or not used. The electrical connector includes two or more lead wires 112, 114 that are designed to be connected to a power source, not shown. The electrical connector is thus used to provide current to the one or more wires in the balloon to cause the wires to heat by resistive heating, which in turn causes the surface of the balloon to be heated as discussed above. FIG. 11 is a cross-sectional view of the balloon catheter between end portion 70 and balloon 40. The balloon catheter includes an outer wall 120 that is generally formed a durable and flexible material. The composition of the outer wall of balloon catheters is well known in the art, thus will not be further discussed herein. The cross-section of the balloon catheter illustrates four passageways; however, it can be appreciated that more than four or less than four passageways can exist in the balloon catheter. Passageway 130 is fluidly connected to port 90 to enable fluid to flow from end portion 70 to balloon 40. Passageway or lumen 140 is connected to guide wire port 80 to enable the guide wire to engage and/or more to the balloon. Passageway 150 is fluidly connected to outlet port 100 to enable fluid to flow from balloon 40 to end portion 70. Passageway or lumen 160 is connected to electrical connector 110 to enable lead wires 112, 114 to connect to the braided wire 50 inside balloon 40.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

1-102. (canceled)

103. A method used in to change the shape of a medical device comprising the steps of:
a. providing a medical device having a first shape, said medical device designed to be implant in an organism, said medical device at least partially formed of a polymeric material, said medical device having a polymeric coating, and combinations thereof;
b. mounting said medical device on a portion of a delivery device;
c. positioning said medical device in a living organism, said medical device at a first temperature when positioned in said living organism;
d. changing a shape of said medical device from said first shape to said second shape while said medical device is positioned in said living organism; and,
e. changing a temperature of said medical device from a first temperature to a second temperature so as to change at least one physical property of said polymeric material, said polymeric coating, and combinations thereof, said step of changing said temperature occurring prior to, during, after, and combinations thereof to said step of changing said shape of said medical device, said step of changing said temperature facilitating in said changing of said shape of said medical device, said delivery device designed to at least partially cause said medical device to change temperature from said first temperature to said second temperature.

104. The method as defined in claim 103, wherein said medical device includes a stent, said delivery device including a catheter, said delivery device designed to provide a heat source to said medical device so as to change said temperature of said medical device from said first temperature to said second temperature, said heat source including heated fluid flowing through said catheter, electrical current flowing through said catheter, and combinations thereof.

105. The method as defined in claim 104, wherein said catheter includes an inflatable balloon designed to at least partially cause said stent to change shape from said first shape to said second shape when said inflatable balloon is inflated, said balloon designed to receive temperature controlled fluid, electrical energy, or combinations thereof to at least partially cause said temperature of said stent to change from said first temperature to said second temperature.

106. The method as defined in claim 105, wherein said catheter includes a central channel and at least one outer channel, said central channel designed to receive a guide wire, said at least one outer channel designed to receive said temperature controlled fluid, at least one electrical wire, and combinations thereof.

107. The method as defined in claim 103, wherein said polymeric material of said medical device, said polymeric coating on said medical device, and combinations thereof has a glass transition temperature, a thermoset temperature, and combinations thereof that is greater than about 40° C.

108. The method as defined in claim 106, wherein said polymeric material of said medical device, polymeric coating on said medical device, and combinations thereof has a glass transition temperature, a thermoset temperature, and combinations thereof that is greater than about 40° C.

109. The method as defined in claim 103, including the step of changing a temperature of said medical device from said second temperature to a third temperature after said medical device has changed shaped from said first shape to said second shape, said delivery device designed to at least partially cause said medical device to change temperature from said second temperature to said third temperature.

110. The method as defined in claim 108, including the step of changing a temperature of said medical device from said second temperature to a third temperature after said medical device has changed shaped from said first shape to said second shape, said delivery device designed to at least partially cause said medical device to change temperature from said second temperature to said third temperature.

111. The method as defined in claim 103, including the step of at least partially curing, at least partially thermosetting, at least partially creating cross-linking, and combinations thereof in said polymeric material of said medical device, polymeric coating on said medical device, and combinations thereof by exposing said polymeric material of said medical device, polymeric coating on said medical device, and combinations thereof to heat, radioactive emissions, light waves, and combinations thereof.

112. The method as defined in claim 110, including the step of at least partially curing, at least partially thermosetting, at least partially creating cross-linking, and combinations thereof in said polymeric material of said medical device, polymeric coating on said medical device, and combinations thereof by exposing said polymeric material of said medical device, polymeric coating on said medical device, and combinations thereof to heat, radioactive emissions, electromagnetic waves, and combinations thereof.

113. A deployment system used to change a shape of a medical device comprising:
a. a medical device having a first shape, said medical device designed to be implant in an organism, said medical device at least partially formed of a polymeric material, said medical device having a polymeric coating, and combinations thereof; and,
b. a delivery device that includes said medical device mounted on a portion of said delivery device, said delivery device including an expansion member designed to change a shape of said medical device from a first shape to a second shape when said medical device is mounted on said delivery device, said delivery device designed to change a temperature of said medical device from a first temperature to a second temperature when said medical device is mounted on said delivery device so as to change at least one physical property of said polymeric material, said polymeric coating, and combinations thereof to facilitate in said changing of said shape of said medical device.

114. The deployment device as defined in claim 113, wherein said medical device includes a stent, said delivery device including a catheter, said delivery device designed to provide a heat source to said medical device so as to change said temperature of said medical device from said first temperature to said second temperature, said heat source including heated fluid flowing through said catheter, electrical current flowing through said catheter, and combinations thereof.

115. The deployment device as defined in claim 114, wherein said catheter includes an inflatable balloon designed to at least partially cause said stent to change shape from said first shape to said second shape when said inflatable balloon is inflated, said balloon designed to receive temperature controlled fluid, electrical energy, or combinations thereof to at least partially cause said temperature of said stent to change from said first temperature to said second temperature.

116. The deployment device as defined in claim 115, wherein said catheter includes a central channel and at least one outer channel, said central channel designed to receive a guide wire, said at least one outer channel designed to receive said temperature controlled fluid, at least one electrical wire, and combinations thereof.

117. The deployment device as defined in claim 113, wherein said polymeric material of said medical device, said polymeric coating on said medical device, and combinations thereof has a glass transition temperature, a thermoset temperature, and combinations thereof that is greater than about 40° C.

118. The deployment device as defined in claim 115, including braided wire, heat conducting band, and combinations thereof on said inflatable balloon, said braided wire, said heat conducting band, and combinations thereof designed to heating said stent when said stent is mounted on said inflatable balloon, said heat conducting band, and combinations attached to, positioned in, positioned about, and combinations thereof an interior portion of said inflatable balloon, an outer surface of said inflatable balloon, and combinations thereof.

119. The deployment device as defined in claim 116, wherein said catheter includes said central channel designed to receive a guide wire, a first outer channel designed to direct said temperature controlled fluid to said inflatable balloon, and a second channel designed to allow said temperature controlled fluid to flow out of said inflatable balloon, said second channel fluidly connected to said central channel or not fluidly connected to said central channel.

120. The deployment device as defined in claim 119, wherein said second channel in said catheter designed to be fluidly connected to said central channel so as to allow said temperature controlled fluid to flow out of said inflatable balloon and into said central channel and be expelled out of a distal end of said catheter.

121. The deployment device as defined in claim 119, wherein said second channel in said catheter designed to not be fluidly connected to said central channel so that said first outer channel and said second channel form a closed loop flow arrangement from said temperature controlled fluid that flows through said inflatable balloon.
